(19) United States
(12) Patent Application Publication
(10) Pub. No.: US 2012/0101420 A1
(43) Pub. Date: Apr. 26, 2012

(54) Dynamic Shoulder Joint Orthesis, in Particular a Shoulder Abduction Orthesis

(76) Inventors: Erich Albrecht, Neubeuern (DE); Hans-Georg Opahle, Rosenheim (DE)

(21) Appl. No.: 13/271,269

(22) Filed: Oct. 12, 2011

(30) Foreign Application Priority Data
Oct. 21, 2010 (DE) 10 2010 049 189.6

(51) Int. Cl. A61F 5/00

(52) U.S. Cl. 602/20

(57) ABSTRACT

In a dynamic shoulder joint orthesis a compensation mechanism is provided between a spring (30) and a support rod (25) to compensate for a change in spring force which occurs when the upper arm splint (9) is pivoted, such that the force to be applied to pivot the upper arm splint (9) in the adduction direction and/or in the abduction direction remains at least approximately the same over the entire pivoting range.

The invention relates to a dynamic shoulder joint orthesis, in particular a shoulder abduction orthesis, according to the preamble of claim 1.

The shoulder joint of an adult human is at risk, in a rather specific manner, of becoming immobile, particularly in terms of abduction, if it does not experience sufficient movement as a result of capsular retraction, particularly in the region of the recessus axillaris, and sticking of the displacement structures. Immobilisation damage to an otherwise healthy shoulder can only be measured after a rest period of approximately one week. Elderly patients are particularly at risk. After a surgical procedure in the case of a lesion of the capsular ligament tissue and gliding structures, the risk of development of a contracture of the shoulder joint is disproportionately higher. However, sufficient movement is extremely important for the function of the shoulder joint in order to avoid contractures. It promotes effusion and oedema resorption and helps to avoid thromboses by accelerating the blood flow.

In a relatively large number of patients, it was determined either during or after a surgical procedure owing to shoulder damage or trauma that there is a considerable restriction of the stability of the structures responsible for abduction of the upper arm. For example, this is the case after a refixing or reconstruction of the tendon of the musculus supraspinatus by sewing, then reattachment to the tuberculum majus, reconstruction by a latissimus dorsi transfer or refixing of the tuberculum majus or with fractures of the proximal humerus, wherein, when treated by means of plates, intermedullary pins or endoprosthesis, the tubercula had to be refixed to the appending tendons of the rotator cuff.

During aftercare, it is generally necessary to protect the reconstructed structures against a renewed rupture or re-dislocation for a relatively long period of time, for example six weeks. The arm is therefore normally immobilised by means of an abduction pad or an abduction orthesis in an abduction position between 30° to 60° (depending on the tone of the refixed tendons determined intraoperatively). In order to keep the above problems caused by insufficient movement to a minimum, exercises are carried out by passive movements of the shoulder, for which the help of an assistant is necessary, for example a physiotherapist or a suitable trained employee. The aftercare may also be supported by the use of a CPM (continuous passive motion) chair. In this case, the arm is laid on a positioning rest of a special treatment chair and moved passively in the shoulder joint in a definable range by means of motor force. However, this method is very cost intensive and involved.

However, owing to the time restriction, the passive movement of a shoulder joint with the aid of a therapist is not sufficiently suitable to replace the movements carried out spontaneously during daily use and to reliably prevent immobilisation damage.

A dynamic shoulder joint orthesis in the form of a shoulder abduction orthesis according to the preamble of claim 1 is further known from DE 84 07 242 U1. This orthesis makes it possible to move the upper arm splint against the resistance of a spring in the adduction or abduction direction over a specific angular range. For this purpose the splint comprises a guide means which can be fastened on the upper body and comprises a housing, in which the lower end of a support rod is displaceably guided. The spring is formed as a compression spring and is increasingly compressed by the lower end of the support rod when the upper arm, for example starting from a horizontally abducted position, is moved downwards in the adduction direction.

With the aid of ortheses of this type it is possible to adjust the orthesis by selecting a suitable spring, in such a way that it holds the upper arm in a specific abduction position or guides it back into this position without the patient having to engage the corresponding muscles. However, a drawback is that the supporting force of the orthesis changes during a pivoting movement of the upper arm. With a movement in the adduction direction, the spring is increasingly compressed, whereby the spring force increases and an increasing muscle force is necessary to move the upper arm downwards into the maximally lowered position. By contrast, the spring initially pushes the upper arm splint out of its lowermost position with a very high spring force in the abduction direction, whilst the spring force decreases with increasing relaxation of the spring. The spring support thus varies widely over the spring path. However, this is particularly undesirable in those cases in which there is a passive mobilisation of the shoulder joint and therefore cooperating tendons and muscles. In contrast to the effect of known abduction ortheses, it may often also be desirable to even increase the supporting force with an increasing angle of abduction of the upper arm, since the weight of the upper arm pressing on the upper arm splint is then greater.

The object of the invention is to provide a dynamic shoulder joint orthesis of the type mentioned at the outset, with which a mobilising treatment of the shoulder is enabled in the most effective and physiologically optimal manner possible.

This object is achieved in accordance with the invention by a dynamic shoulder joint orthesis having the features of claim 1. Advantageous embodiments of the invention are described in the further claims.

In the shoulder joint orthesis according to the invention a compensation mechanism is provided between the spring and the support rod and counteracts the change in spring force which occurs when the upper arm splint is pivoted, such that the force to be applied to pivot the upper arm splint in the adduction direction and/or in the abduction direction remains at least approximately the same over the entire pivoting range.

The shoulder joint orthesis according to the invention enables a very effective mobilising treatment of the shoulder joint. If the joint is released in the range of motion deemed to be favourable by the operator and the spring force is set so that the weight of the arm supported thereon is overcome, in particular an independent mobilisation of the shoulder joint which is load-free for the adductors can be carried out by the patient. In particular it is possible that, owing to a low engagement of the generally unimpaired adductors of the shoulder (M. Latissimus dorsi, M. Pectoralis major, M. Subscapularis, M. Teres major and minor), the arm is adducted in the shoulder joint as far as a defined starting point (for example a maximum of 30°) and once the adductors relax is abducted via the spring force as far as the defined end point (generally 90°).

In this instance the “Kleinert principle” is implemented, that is to say when the agonists are activated the antagonists are relaxed by way of reflex. In this case, when the adductors are engaged, the abductors are relaxed and the abduction is then carried out by the spring force such that the structures in need of protection during the movement of the shoulder joint always remain free from tensile loading and their healing is not disturbed.

A particular advantage is that this passive mobilising of patients, which is load-free for the abductors of the shoulder, can be carried out at any time without further technical or human aid.

If there is already an abduction constraint of the shoulder joint (abduction contracture) caused by a sticking and scarring of the recessus axilaris and the tendons, it is possible to effect a forced abduction by a corresponding setting of the spring force significantly above the weight of the arm. A gradual expansion of the shoulder and therefore an increase in the range of motion can thus be achieved within the sense of a passive mobilisation treatment.

In accordance with an advantageous embodiment the compensation mechanism comprises a cam which is rotatable about an axis of rotation and can be coupled in motion to a force transfer element, preferably a Bowden cable, cooperating with the lower end of the support rod, wherein a lever acts between the force transfer element and the axis of rotation of the cam, the length of which lever becomes greater with increasing spring force. In this embodiment the length of the effective lever arm, over which a torque is exerted on the cam by the force transfer element, thus changes depending on the rotational position of the cam. For example, if a constant force is to be exerted on the lower end of the support rod over the entire path of displacement, the effective lever length over which the force transfer element exerts a torque on the cam can be increasingly enlarged with an increasing angle of rotation of the cam and therefore also with increasing spring force, whereby the torque exerted on the cam by the force transfer element is increased to the same extent as the torque which the spring exerts on the cam.

Of course, it is also possible to set the compensation mechanism in such a way that the displacement force transferred by the spring onto the lower end of the support rod does not remain constant over the entire path of displacement, but if necessary adapts to the weight of the upper arm which is momentarily acting on the upper arm splint and changes depending on the angular position. In particular it is possible to set the displacement force transferred by the spring onto the lower end of the support rod in such a way that it corresponds at any point of the path of displacement to the force which is necessary to overcome the weight of the arm in any adduction or abduction position of the upper arm and to thus enable a movement of the arm without nominal loading of the muscles.

In accordance with an advantageous embodiment the force transfer element comprises a Bowden cable which is guided over a peripheral surface portion of the cam, wherein the distance between the peripheral surface portion and the axis of rotation changes along the periphery of the cam. This enables a simple, space-saving and very reliably functioning design of the orthesis.

The spring advantageously consists of a spiral spring, of which the primary plane is arranged parallel to the primary plane of the cam. A very space-saving and simple design can thus likewise be achieved.

In accordance with an advantageous embodiment the spiral spring is coupled at one end to a gearwheel in a rotationally engaged manner, which gearwheel is mounted rotatably in the housing of the guide means to adjust the biasing force of the spiral spring. The biasing force of the spring can thus be changed in a very simple manner and adapted to the respective requirements without having to change the spring.

The gearwheel is advantageously rotatable by means of a worm wheel which is rotatable via a manually actuatable rotation mechanism. A self-locking gear between the worm wheel and the gearwheel can thus be provided, which maintains the set position of the gearwheel without further locking elements.

A very precise working orthesis and accurate guidance of the support rod are provided if a sliding carriage is guided in the housing of the guide means in a longitudinally displaceable manner, to which sliding carriage the lower end of the support rod is articulated and to which the Bowden cable is fastened in such a way that the sliding carriage is coupled in motion both with the upper arm splint and with the spring-loaded cam during an abduction or adduction movement of the upper arm splint.

The pivoting range of the upper arm splint is advantageously defined by a sliding carriage path delimitation mechanism which comprises stops arranged in or on the housing and is adjustable in the longitudinal direction of the slide path. For this purpose, the sliding carriage path delimitation mechanism expediently comprises two parallel spindles arranged in the housing which each carry a stop in the form of a spindle nut. As a result, the associated stop can be adjusted in a simple manner by turning a corresponding spindle so as to accordingly change the pivoting range of the upper arm splint.

The invention will now be described in greater detail and by way of example on the basis of the drawings, in which:

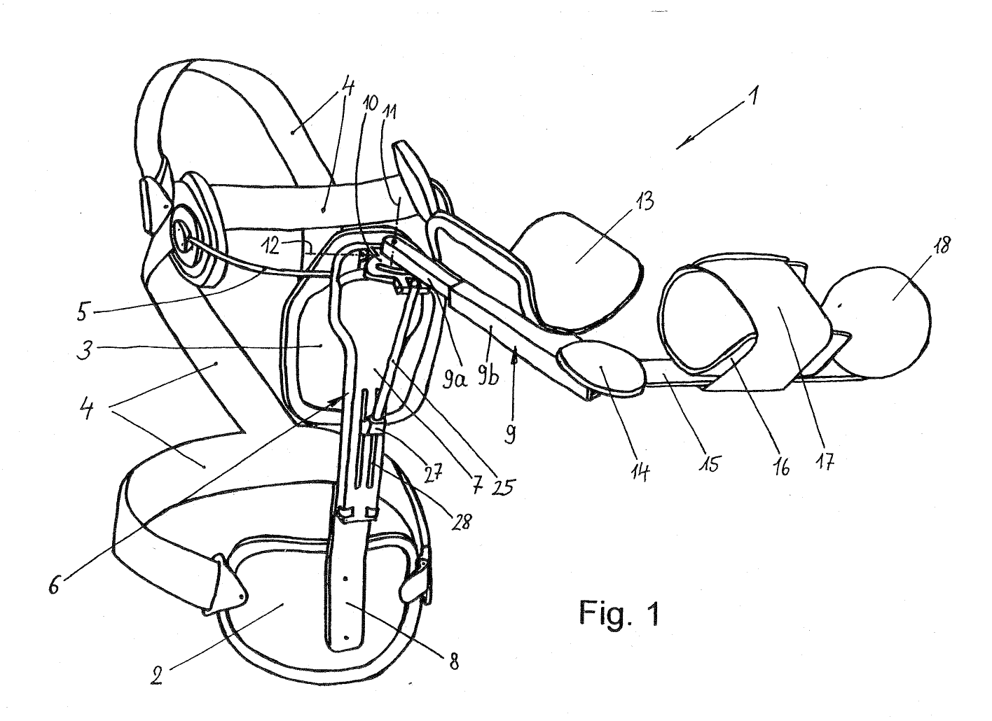

FIG. 1: is a three-dimensional view of a shoulder joint orthesis according to the invention;

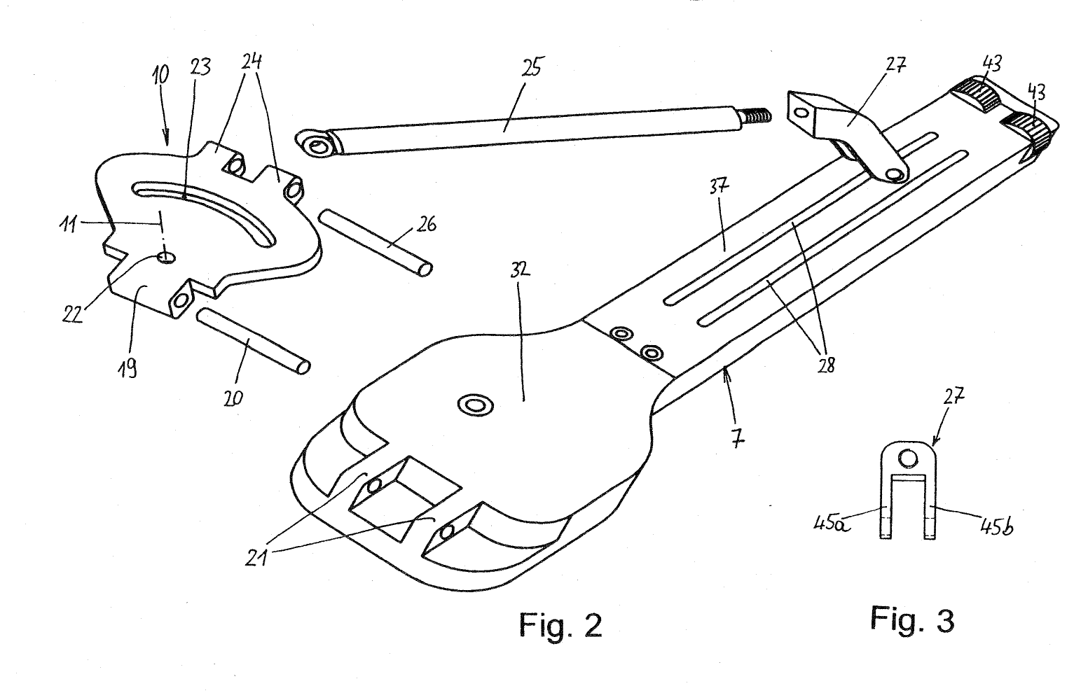

FIG. 2: is an exploded view of the guide means, support rod, hinge plate and the support rod coupling part;

FIG. 3: is a front view of the coupling part of FIG. 2;

FIG. 4 shows the main individual parts of the guide means;

FIG. 5: shows the individual parts of FIG. 4 in the assembled state;

FIG. 6: is a longitudinal sectional view through the guide means of FIG. 5;

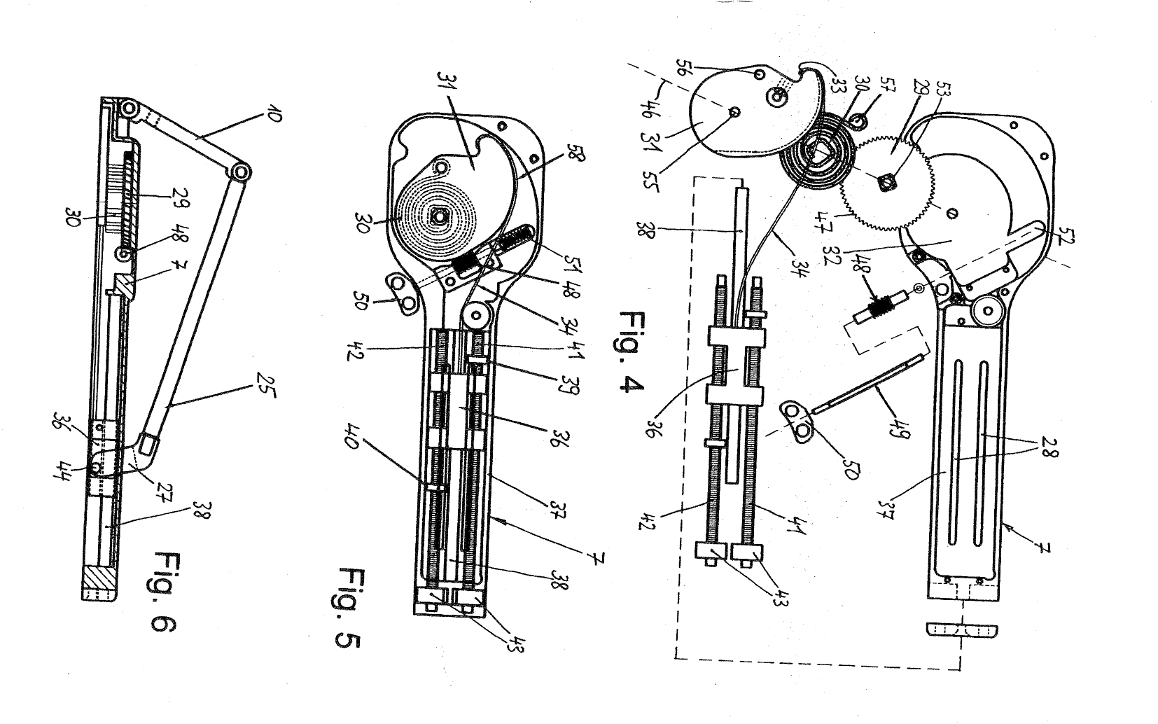

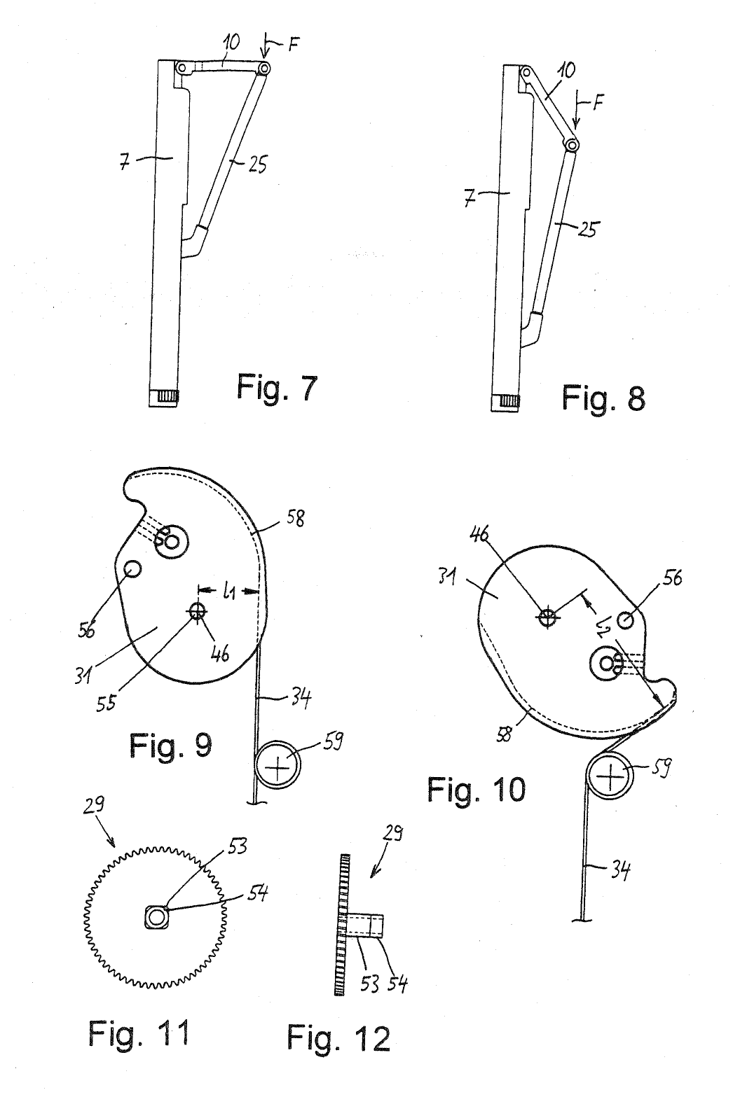

FIG. 7: is a side view of the guide means, wherein the support rod and hinge plate are in a first, raised position;

FIG. 8: is an illustration according to FIG. 7, wherein the support rod and hinge plate are in a second, lowered position;

FIG. 9: shows the cam and the Bowden cable in a rotational position of the cam which it adopts with a position of the orthesis according to FIG. 7;

FIG. 10: shows the cam and the Bowden cable when the orthesis is in the position of FIG. 8;

FIG. 11: is a front view of the gearwheel of the guide means; and

FIG. 12: is a side view of the gearwheel of FIG. 11.

The shoulder joint orthesis 1 illustrated in FIG. 1 comprises a lower support element 2, which rests laterally against the upper body in the hip region, and an upper contact element 3, which rests laterally against the chest directly beneath the shoulder. The support element 2 and contact element 3 are fastened to the upper body by means of a harness which, in the embodiment illustrated, comprises a belt 4 and a stable steel clasp 5.

The support element 2 and contact element 3 fix a stable guide means 6 which comprises an elongate housing 7 and an extension bar 8 fixed thereto. The housing 7 and extension bar 8 expediently consist of metal, for example aluminium. In the assembled state of the shoulder joint orthesis 1, the longitudinal axis of the housing 7 and of the extension bar 8 extend substantially vertically, wherein the housing 7 is fastened to the upper contact element 3 and extends as far as the vicinity of the shoulder, that is to say as far as the vicinity of the patient's armpit area, whereas the longitudinal bar 8 is fastened to the lower end of the housing 7 and to the lower portion element 2 and, depending on length, determines the distance between the support element 2 in the vicinity of the hip and the upper contact element 3.

In order to hold the arm of a patient in the abducted position and/or to guide it in a supported manner in the abduction and adduction directions, an upper arm splint 9 is mounted in an articulated manner in the upper end region of the housing 7 by means of a hinge plate 10. In order to adjust the length of the upper arm splint 9, said splint consists of two splint parts 9a, 9b which are guided inside one another in a telescope-like manner and can be displaced relative to one another so as to vary the extension length. The upper arm splint 9 is also pivotable at one end relative to the hinge plate 10 about a pivot axis 11, so that the upper arm splint 9 can be fixed to the hinge plate 10 at different angles. A circular arc-shaped slot 23 is provided in the hinge plate 10 for this purpose, through which slot a screw (not shown) can be guided, with which the proximal splint part 9a is fixed to the hinge plate 10. The pivot axis 11 extends perpendicular to the pivot axis 12 about which the hinge plate 10 can be pivoted relative to the housing 7 in the abduction and adduction directions.

A half-shell-shaped upper arm support 13 is fixed to the upper face of the splint part 9b, in which support the upper arm can be laid. When the distal splint part 9b is displaced relative to the proximal splint part 9a, the upper arm support 13 is thus accordingly entrained, so that the distance between the upper arm support 13 and the orthesis articulation can be changed.

The upper arm splint 9 is connected at its distal end via an articulation arranged beneath a pad 14 to a lower arm splint 15. A half-shell-shaped lower arm support 16 is fixed in said lower arm splint, in which support the lower arm can be laid. The lower arm can be fixed in the lower arm support 16 by means of a fastening strip 17. A hand support 18, which in particular may take the form of a round or spherical pad which enables the patient to carry out kneading exercise with his fingers, is located at the distal end of the lower arm splint 15.

The hinge plate 10 forms a living hinge with the guide means 6 and for this purpose comprises at one end a hinge tab 19 (FIG. 2) which is connected in an articulated manner to bearing webs 21 of the housing 7 via a hinge pin 20. In order to mount the upper arm splint 9 so as to be pivotable relative to the hinge plate 10 about the pivoting axis 11, a hole 22 is provided in the vicinity of the hinge tab 19, into which hole a hinge pin (not shown) is introduced.

The upper arm splint 9 is fixed in the proximal end region of the splint part 9a to the hinge plate 10. A corresponding pivoting of the hinge plate 10 in the adduction and abduction directions is thus coupled with a corresponding pivoting of the upper arm splint 9 and thus also of the lower arm splint 15.

The hinge plate 10 further comprises, at its end opposite the hinge tab 19, two hinge tabs 24 which connect the upper end of a support rod 25 in an articulated manner. For this purpose, the support rod 25 is introduced via its upper end between the hinge tabs 24 and fixed by means of a hinge pin 26.

The upper arm splint 9 and therefore the patient's arm lying thereon is held in the desired abduction positions relative to the upper body by means of support rods 25 or exerts a support force from beneath onto the hinge plate 10 and therefore onto the upper arm splint 9 during corresponding abduction and adduction movements. The lower end of the support rod 25 engages via a prong-shaped coupling element 25 in two parallel longitudinal slits 28 in the housing 7 and can be displaced along the longitudinal slit 28 either against the action or with the assistance of a spring mechanism which will be described below in greater detail. The spring force can be set in such a way that the support force acting on the upper arm splint 9 compensates for the weight of the patient's arm in any abduction position of the arm such that the arm can be moved weightlessly, that is to say without any nominal active muscular support, in the abduction direction. Furthermore, it is also possible to set the spring force so that the shifting force applied by the spring mechanism and acting on the support rod 25 is greater than the weight of the arm including the arm splint so that an active passive mobilisation treatment of the shoulder joint is enabled in the abduction direction. Further, it is possible to fix the support rod 25 in any desired position of the range of displacement, that is to say a continuous fixing of the upper arm splint 9 and therefore a static fixing of the arm in any abduction position is possible.

The structure and operating principle of the guide means 6 will be described hereinafter in greater detail with reference to FIGS. 4 to 6.

The guide means 6 comprises the housing 7 as well as a gearwheel 29, a spring 30 in the form of a spiral spring, and a cam 31 which are housed in a head part 32 of the housing 7. One end 33 of a cable 34 is fastened to the cam 31, the other end of said cable being fastened to a sliding carriage 36.

The sliding carriage 36 is arranged displaceably in an elongate housing portion 37 which connects to the head part 32. The sliding carriage 36 is guided by a guide rod 38 which is arranged centrally in a stationary manner in the housing portion 37 and which is guided, with little play, through a corresponding hole extending longitudinally in the sliding carriage 36. The range of displacement of the sliding carriage 36 along the housing portion 37 is defined by stops 39, 40 which are formed as spindle nuts. Each stop 39, 40 is displaceable by its own spindle 41, 42 in the form of threaded bars arranged parallel to one another on either side of the guide bar 38 in the housing portion 37. Since the stops 39, 40 are arranged in the housing 7 in a non-rotationally-engaged manner, a rotation of the spindles 41, 42 displaces the stops 39, 40 in the longitudinal direction of the housing 7. The spindles 41, 42 are rotated via knurled discs 43 which are connected so as to be rotationally engaged and protrude beyond the housing 7 at the end of the guide portion 37 (see FIG. 2 also).

In the embodiment illustrated the stop 39 is located on the side of the sliding carriage 36 facing the head part 32, whereas the stop 40 is located on the opposite side of the sliding carriage 36. In FIG. 5 the stop 39 thus defines the path of displacement of the sliding carriage 30 to the left, that is to say upwardly in FIG. 1, and therefore the pivoting range of the upper arm splint 9 in the abduction direction. By contrast, the stop 40 defines the path of displacement of the sliding carriage 36 in FIG. 5 to the right, that is to say downwardly in FIG. 1, and therefore the pivoting range of the upper arm splint 9 in the adduction direction.

The coupling element 27 is connected in an articulated manner to the sliding carriage 36 via a cross-bolt 44 (FIG. 6). In this case the two side branches 45a, 45b (FIG. 3) of the coupling element 27 penetrate the longitudinal slits 28 in the housing 7. A displacement of the sliding carriage 36 in the longitudinal direction of the housing 7 thus accordingly displaces the lower end of the support rod 25.

The sliding carriage 36 is displaced either against or with the assistance of the spring force of the spring 30 depending on whether the upper arm splint 9 is moved in the adduction direction or in the abduction direction. For this purpose, the spring 30 applies a permanent tensile force on the sliding carriage 36 in the direction of the head part 32 via the cam 31 and the cable 34. A cable guide roll 59 which is mounted rotatably in the housing 7 in the region between the cam 31 and the sliding carriage 36 and can be seen in FIGS. 4, 5, 9 and 10 ensures that the cable 34 is fed parallel to the direction of displacement of the sliding carriage 36 in the housing portion 37.

The biasing force of the spring 30 is set via the gearwheel 29. The gearwheel 29 is rotatable about an axis of rotation 46 which extends transversely (FIG. 4) and is determined by a cross-bolt (not shown) mounted in the head part 32. On its periphery the gearwheel 29 carries an end-face toothing 47 which cooperates with a worm wheel 48. The worm wheel 48 is rotatably connected to a shaft 49 which penetrates the worm wheel 48 in the longitudinal direction. The shaft 49 and therefore the worm wheel 48 can be rotated via a leaf-shaped handling part 50 which is attached to the outer end of the shaft 49. By rotating the worm wheel 48 by means of the shaft 49, the gearwheel 29 is thus accordingly rotated about the axis of rotation 46. Owing to the self-locking property of the worm gear pair, the set position of rotation of the gearwheel 29 relative to the housing 7 is maintained.

It can further be seen from FIG. 5 that a compression spring 51 is provided on the inner end of the shaft 49, which compression spring is received in a recess 52 (FIG. 4) of the housing 7. In the actuation position shown in FIG. 5, the inner end of the shaft 49 extends only slightly into the compression spring 51 and is supported axially towards the compression spring 51. It is thus possible to push the shaft 49, together with the handling part 50, axially into the housing 7 against the compressive force of the compression spring 51 during non-use, such that the handling part 50 no longer protrudes beyond the housing 7.

So that this displacement can take place, the worm wheel 48 mounted undisplaceably in the head part 32 is mounted displaceably, yet non-rotationally on a central polygonal portion, for example a square portion of the shaft 49.

The pushed-in position of the shaft 49 is retained by means of a locking mechanism (not shown). In order to actuate the shaft 49, the locking mechanism is released, whereby the compression spring 51 displaces the shaft 49 together with the handling part 50 into the position shown in FIG. 5.

As can be seen from FIGS. 4, 11 and 12, the gearwheel 29 comprises a central, axial protruding square lug 53 and a cylindrical lug 54 connecting thereto. The inner, likewise square curved end of the spring 30 is placed on the square lug 53 and said spring is thus connected to the gearwheel 29 in a rotationally engaged manner. The cam 31 is placed on the cylindrical lug 54 of the gearwheel 29, wherein the cylindrical lug 54 penetrates a bore 55 in the cam 31. The cam 31 is therefore rotatable relative the gearwheel 29 about the axis of rotation 46.

The cam 31 is coupled to the spring 30 by means of a driving bolt 56 which engages in an eye 57 at the outer end of the spring 30. Owing to the biasing force of the spring 30, a torque is thus exerted in the cam 31 about the axis of rotation 46, which torque attempts to rotate the cam 31 in an anti-clockwise direction in the embodiment shown in FIGS. 4 and 5. Since the cable 34 is fastened at one end to the cam 31 and is guided along the periphery, that is to say over a peripheral surface portion 58, of the cam 31, said cam tensions the cable 34 and thus the sliding carriage 36.

It can be seen that an abduction movement of the arm is assisted by the force with which the sliding carriage 36 is pulled upwardly by means of the spring mechanism, whereas with an adduction movement of the arm the spring force acts as a braking force since the sliding carriage 36 is displaced downwardly against the spring force.

With an adduction movement of the arm, the spring force would normally increase with an increasing path of displacement of the sliding carriage 36 since the spring 30 is increasingly deformed. By contrast, with an abduction movement of the arm the spring support would decrease with increasing relaxation of the spring 30. In order to avoid these undesired effects, the cam 31 is specially shaped, thus forming a compensation mechanism which counteracts the change in spring force in such a way that the force to be applied to move the upper arm splint 9 remains at least approximately the same along the path of displacement of the support rod 25. For this purpose the cable 34, as can be seen in FIGS. 9 and 10, extends over a peripheral surface portion 48 of the cam 31, the distance of which to the axis of rotation 46 of the cam 31 changes along the periphery of the cam 31. In FIGS. 9 and 10 the peripheral surface portion 48 with which the cable 34 is more or less in contact depending on the rotational position of the cam 31 is indicated by a dashed line, since the cam 31 comprises a guide groove in this region, in which groove the cable 34 is recessed.

The rotational position of the cam 31 illustrated in FIG. 9 corresponds to that which is adopted in a maximally raised position of the upper arm splint 9 or of the hinge plate 10, as shown in FIG. 7. This position corresponds, for example, to a 90° position relative to the housing 7. In this position the support rod 25 is located in its highest position, wherein the spring 30 is relaxed to the maximum. The torque which is applied via the spring 30 to the cam 31 is thus relatively low owing to the low spring force.

As can be seen from FIG. 9, however, the effective lever arm 1₁ is also relatively short between the axis of rotation 46 and the region of the peripheral surface portion 48 over which an opposed torque is applied to the cam 31 when the cable 34 is tensioned. This short lever arm 1, thus also exerts a relatively low counter-torque on the cam 31 by the weight F acting on the upper arm splint 9 (FIG. 7).

If, as can be seen from FIG. 8, the arm is now moved downwardly together with the hinge plate 10 and the support rod 25, the sliding carriage 36 is also displaced downwardly and the cam 31 is rotated via the cable 34 in a clockwise direction into the position illustrated in FIG. 10. The cable 34 thus remains in contact with an increasingly smaller part of the peripheral surface portion 58, wherein the peripheral surface portion 58 is distanced increasingly further from the axis of rotation 46. With the maximum rotation of the cam 31 shown in FIG. 10, the effective lever arm 1₂, via which the cable 34 exerts a counter-torque on the cam 31, is longer than the effective lever arm 1₁ of FIG. 9. If tensile force is applied to the cable 34, a greater counter-torque can thus be transferred to the cam 31 by the extended lever arm 1₂, said counter-torque counteracting the greater torque which is caused by the increasing deformation of the spring 30 and is applied to the cam 31 by the spring 30. The increasing spring force is thus compensated for by an effective lever arm which becomes longer.

The curve of the peripheral surface portion 58 is formed in such a way that the described compensation of the change in spring force is achieved by changing the effective lever lengths 11, 12 in any angular position of the upper arm splint 9. If desired, the upper arm can thus be moved weightlessly over the entire pivoting range in adduction and abduction directions.

By changing the biasing force of the spring 30 by rotating the gearwheel 29, the force with which the patient's arm is supported can be changed continuously. As healing progresses, the spring force can thus also be increasingly reduced so that the patient has to increasingly load the muscles required for abduction. By contrast, the spring force can be increased so that the patient's arm is pushed upwardly by the upper arm splint 9 so as to passively mobilise and expand the shoulder joint. There are thus many fields of application of the shoulder joint orthesis according to the invention.

1. Dynamic shoulder joint orthesis, in particular a shoulder abduction orthesis, comprising:
a guide means which can be fastened on the upper body and comprises a housing,
an upper arm splint which is fastened in an articulated manner to the guide means and can be moved at least in the adduction and abduction directions,
a support rod for supporting the upper arm splint, the support rod comprising a lower end which is guided displaceably on the guide means,
a spring for applying a displacement force on the lower end of the support rod
wherein a compensation mechanism is provided between the spring and the support rod and counteracts a change in spring force which occurs when the upper arm splint is pivoted, such that the force to be applied to pivot the upper arm splint in the adduction direction and/or in the abduction direction remains at least approximately the same over the entire pivoting range.

2. Shoulder joint orthesis according to claim 1, wherein the compensation mechanism comprises a cam which is rotatable about an axis of rotation and can be coupled in motion to a force transfer element cooperating with the lower end of the support rod, a lever arm acting between the force transfer element and the axis of rotation of the cam, the length 1₁, 1₂ of which lever becomes greater with increasing spring force.

3. Shoulder joint orthesis according to claim 2, wherein the force transfer element comprises a Bowden cable which is guided over a peripheral surface portion, the distance between the peripheral surface portion and the axis of rotation changing along the periphery of the cam.

4. Shoulder joint orthesis according to claim 2, wherein the spring consists of a spiral spring which is arranged in terms of the primary plane parallel to the primary plane of the cam.

5. Shoulder joint orthesis according to claim 4, wherein the spiral spring is coupled at one end to a gearwheel in a rotationally engaged manner, which gearwheel is mounted rotatably in the housing of the guide means to adjust the biasing force of the spiral spring.

6. Shoulder joint orthesis according to claim 5, wherein the gearwheel and the cam are arranged parallel to one another and are rotatable about the same axis of rotation.

7. Shoulder joint orthesis according to claim 5, wherein the gearwheel is rotatable by means of a worm wheel which is rotatable by a manually actuatable rotation mechanism.

8. Shoulder joint orthesis according to claim 3, wherein a sliding carriage is guided in the housing of the guide means in a longitudinally displaceable manner, to which sliding carriage the lower end of the support rod is articulated and to which the Bowden cable is fastened in such a way that the sliding carriage is coupled in motion both with the upper arm splint and with the spring-loaded cam during an abduction or adduction movement of the upper arm splint.

9. Shoulder joint orthesis according to claim 8, wherein the pivoting range of the upper arm splint is defined by a sliding carriage path delimitation mechanism which comprises stops which are arranged in or on the housing and are adjustable in the longitudinal direction of the sliding carriage path.

10. Shoulder joint orthesis according to claim 9, wherein the sliding carriage path delimitation mechanism comprises two spindles which are arranged in parallel in the housing and which each bear a stop in the form of a spindle nut.
